(19) United States
(12) Patent Application Publication
(10) Pub. No.: US 2026/0020907 A1
(43) Pub. Date: Jan. 22, 2026

(54) APPARATUSES AND METHODS FOR TEMPERATURE MEASUREMENT AT ABLATION PROBE HANDLE

(71) Applicant: Varian Medical Systems, Inc., Palo Alto, CA (US)

(72) Inventors: Justin May, Austin, TX (US); Steve Jester, Eden Prairie, MN (US); Jomar Ochoco, St. Paul, MN (US); David Weinzierl, Andover, MN (US); Dane Olson, New Hope, MN (US)

(21) Appl. No.: 18/778,462

(22) Filed: Jul. 19, 2024

(51) Int. Cl. A61B 18/18; A61B 18/00

(52) U.S. Cl. CPC A61B 18/1815; A61B 2018/00023; A61B 2018/00577; A61B 2018/00666; A61B 2018/00821; A61B 2018/00898; A61B 2018/00988; A61B 2018/1869; A61B 2560/0228

(57) ABSTRACT

A microwave ablation probe includes a handle configured to operably couple a supply cable to a needle and a printed circuit board assembly (PCBA) positioned in the handle. The PCBA includes an analog to digital converter and an electrically erasable programmable read-only memory (EEPROM) device.

### TECHNICAL FIELD

The disclosure relates to microwave ablation probes. More specifically, the disclosure relates to microwave ablation probes that may determine or measure one or more temperatures at or in the ablation probe handle.

### BACKGROUND

Microwave ablation probes can be used in clinical treatments such as thermal ablation treatments. In such treatments, thermal ablation can be used to destroy undesirable tissue such as malignant cells in a body. A microwave ablation antenna can be included in the probe and be used to deliver Radio Frequency (RF) energy such as microwave energy to a target tissue to heat the target tissue and destroy the target tissue. The microwave ablation antenna can be positioned inside the ablation probe that can position the microwave ablation antenna proximate the target tissue.

It may be desirable to determine a temperature at one or more locations in a handle of the microwave ablation probe or at one or more locations along a needle of the microwave ablation probe. The temperature at various locations may be used to operate the microwave ablation probe to achieve a desired ablation zone that destroys abnormal cells at the target tissue while minimizing damage to healthy tissues and/or surrounding body structures. Existing apparatuses and methods suffer from drawbacks that may include poor signal strength and/or poor measurement accuracy. Furthermore, existing apparatuses and methods may be expensive and may be susceptible to damage. There exists a need, therefore, for improved microwave ablation probes that overcome the drawbacks of existing apparatuses and methods.

### SUMMARY

The present disclosure is directed to microwave ablation probes that may include a printed circuit board assembly (PCBA) in the handle of the probe. The PCBA may allow various functionality to be performed in the handle and/or using the elements of the PCBA to improve functionality over existing microwave ablation probes. In some embodiments, the PCBA may include an analog to digital converter that may convert analog signals from thermocouples or other sensors to digital signals before such signals are transmitted to an ablation console or other computing device. In some embodiments, the PCBA may include an electrically erasable programmable read-only memory (EEPROM) device. The EEPROM device may allow information to be stored in the ablation probe that can be accessed and used to determine usage, testing performed, designated patient, manufacturing, or other information.

In accordance with some embodiments, a microwave ablation probe may include a handle configured to operably couple a supply cable to a needle, and a printed circuit board assembly (PCBA) positioned in the handle. The PCBA may include an analog to digital converter and an electrically erasable programmable read-only memory (EEPROM) device.

In one aspect, the PCBA may be operatively coupled to a plurality of thermocouples positioned at a plurality of locations in the microwave ablation probe.

In another aspect, the plurality of thermocouples may include a first thermocouple located in the handle and configured to determine a temperature of a supply coolant, and a second thermocouple located in the handle and configured to determine a temperature of a return coolant.

In another aspect, the plurality of thermocouples may include a third thermocouple located in or on the probe needle and configured to determine a temperature of the probe needle at or near the ablation zone.

In another aspect, the analogue to digital converter may be configured to receive a plurality of analog temperature signals from the plurality of thermocouples and send a digital temperature signal based on the plurality of analog temperature signals.

In another aspect, the microwave ablation probe may include: a plurality of thermocouple wires each operably connecting a respective thermocouple of the plurality of thermocouples to the PCBA, and a digital signal wire operably connecting the PCBA to an ablation console.

In another aspect, the EEPROM device may be configured to store probe information characterizing a treatment performed using the microwave ablation probe.

In another aspect, the EEPROM device may be configured to store calibration information characterizing a calibration of or testing performed for the microwave ablation probe.

In some embodiments of the present disclosure, a microwave ablation apparatus is provided. The microwave ablation apparatus may include: at least one of the microwave ablation probes of the present disclosure, an ablation console comprising at least one computing device, and the supply cable operably connecting the microwave ablation probe to the ablation console.

In one aspect, the at least one computing device may be configured to: obtain a coolant temperature signal from the PCBA characterizing a temperature of a coolant in the handle, and send a coolant temperature alert when the temperature exceeds a predetermined coolant temperature threshold.

In another aspect, the coolant temperature signal may be a digital signal obtained from the analog to digital converter.

In another aspect, the at least one computing device may be configured to: obtain a needle temperature signal from the PCBA characterizing a temperature of the needle, and send a needle temperature alert when the temperature exceeds a predetermined needle temperature threshold.

In another aspect, the needle temperature signal may be a digital signal obtained from the analog to digital converter.

In another aspect, the at least one computing device may be configured to: obtain usage information from the EEPROM device, compare the usage information to treatment information, and send a usage alert when the usage information does not correspond to the treatment information.

In another aspect, the at least one computing device may be configured to: prevent usage of the microwave ablation probe when the usage information does not correspond to the treatment information.

In some embodiments of the present disclosure, a method of monitoring a performance of a microwave ablation probe is provided. The method may include obtaining an analog temperature signal from a microwave ablation probe, converting the analog temperature signal to a digital temperature signal in the handle of the microwave ablation probe, and sending the digital temperature signal to a microwave ablation console.

In one aspect, the analog temperature signal may characterize a temperature of coolant in the handle of the microwave ablation probe.

In another aspect, the analog temperature signal may characterize a temperature of a probe needle at or near the ablation zone.

In another aspect, the method may include sending usage information from an electrically erasable programmable read-only memory (EEPROM) device located in the handle of the microwave ablation probe.

In another aspect, the method may include sending a temperature alert when the temperature exceeds a predetermined temperature threshold.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The features and advantages of the present disclosures will be more fully disclosed in, or rendered apparent by the following detailed descriptions of example embodiments. The detailed descriptions of the example embodiments are to be considered together with the accompanying drawings wherein like numbers refer to like parts and further wherein:

FIG. 1 is an isometric view of an example microwave ablation apparatus in accordance with some embodiments of the present disclosure.

FIG. 2 is an example microwave ablation probe that may include a printed circuit board assembly (PCBA) in accordance with some embodiments of the present disclosure.

FIG. 3 is a side view an example microwave ablation handle with a top portion of the housing removed in accordance with some embodiments of the present disclosure.

FIG. 4 is isometric transparent view of the handle of FIG. 3.

FIG. 5 is a flow chart illustrating an example method of monitoring a performance of a microwave ablation probe in accordance with some embodiments of the present disclosure.

FIG. 6 is a block diagram illustrating an example computing device that may be used in some embodiments of the present disclosure.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The microwave ablation probes of the present disclosure may include a handle connected to a needle. The handle may include a printed circuit board assembly (PCBA) positioned inside a housing of the handle. The PCBA may include an analog to digital converter and an electrically erasable programmable read-only memory (EEPROM) device.

The PCBA that is positioned in the handle of the microwave ablation probes of the present disclosure allows improved functionality over existing and/or traditional microwave ablation probes. The PCBA may include an analog to digital converter that may allow analog signals from thermocouples to be converted to digital signals that can be more easily and accurately transmitted to an ablation console. In existing probes, the analog signal from a thermocouple or temperature sensor may be transmitted in analog from the thermocouple, to the ablation console. This requires a suitable length of wire to allow the accurate transmission of the signal without signal loss. Furthermore, the analog signals are susceptible to noise, interference, and distortion that may impact the accuracy and/or reliability of the analog signals. The probes of the present disclosure convert the analog signal to a digital signal in the handle of the probe and then transfer the digital signal to the ablation console. This results in improved signal quality, accuracy, and reliability.

The microwave ablation probes of the present disclosure may also include an EEPROM in the probe handle. Information may be stored and accessed to provide information to the user of the probe. Existing probes do not include such functionality. As such, the use of existing probes may be susceptible to human error if a previously used probe is attempted to be used in a subsequent treatment or the incorrect probe is selected by the user. The EEPROM may provide information to the user that can indicate that the probe was previously used or if the probe does not match characteristics of the probe required for the current treatment.

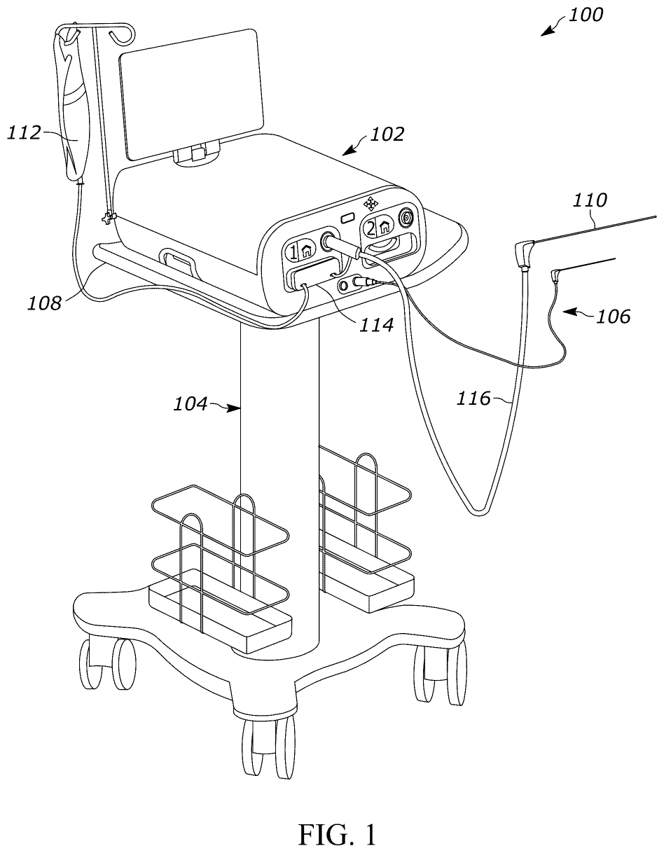

Turning now to FIG. 1, an example microwave ablation apparatus 100 is shown. In this example, the ablation apparatus 100 includes an ablation console 102, and an ablation probe 110. The ablation console 102 may be portable, in some examples, and may be positioned on a platform 108 of stand 104 to allow the console 102 to be easily moved and positioned as desired during treatment. The console 102 may include a microwave generator that may provide a power signal that is sent to an antenna in the needle of the ablation probe 110. The power signal may be provided to the antenna via a supply cable 116 that may extend from the console 102 to the ablation probe 110. The supply cable 116 may also provide a coolant to the ablation probe 110. The coolant may be a saline solution or other coolant that may be supplied to the ablation probe 110 from a coolant receptacle 112 via a pump cartridge 114 or other pump mechanism.

During an ablation treatment, the needle of the ablation probe 110 may be positioned at or near a target tissue in a patient. A power signal may be supplied to the ablation probe 110 from the console 102. The power signal may be supplied to the ablation probe 110 via the supply cable 116. The power signal may cause microwaves to be emitted from an antenna in the needle of the ablation probe 110. The microwaves may cause the tissue at the needle to be heated to a suitable temperature to destroy the target tissue. A temperature sensor 106 may be positioned at or near the target tissue to measure a temperature that may be achieved at or near the ablation zone. In addition and as further described below, the needle or the handle of the ablation probe 110 may include other thermocouples or temperature probes to provide information regarding temperatures of the coolant or the needle.

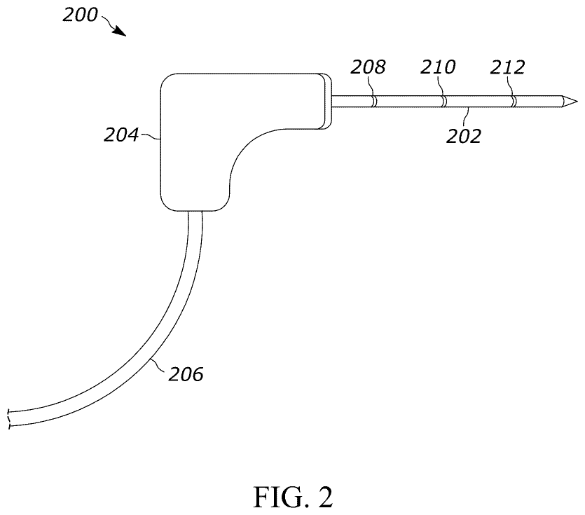

Referring now to FIG. 2, an example ablation probe 200 is shown. The ablation probe 200 may be used with the microwave ablation apparatus 100 previously described. The ablation probe 200 may include a needle 202, a handle 204, and a supply cable 206. The ablation probe 200 may be a disposable or single-use probe that is used during a single procedure or for a single patient. In other examples, the ablation probe 200 (or portions of the ablation probe 200) may be re-used.

The needle 202 of the ablation probe 200 may be an elongated member that maybe inserted into a patient at or near the target tissue. The needle 202 may be a metal, alloy or other rigid material. The needle 202 may be cylindrical and pointed. The needle 202 may be hollow such that various elements are positioned inside the needle 202. For example, a power line and an antenna may be located inside needle 202. The power line may be positioned inside the needle 202 to deliver a power signal from a microwave generator to the antenna. Coolant lines may also be positioned inside the needle to deliver coolant to a distal end of the needle 202. The coolant may be used to control a temperature of the needle 202 and/or to control a size and shape of the ablation zone that may be created at the distal end of the needle 202.

The handle 204 may connect and/or operably couple aspects of the ablation probe 200 that are provided from the supply cable 206 to the needle 202. The supply cable 206 may include a power line that delivers the power signal from the microwave generator. The supply cable 206 may also include a coolant supply line and a coolant return line. The coolant supply line may deliver coolant to the needle 202 and the coolant return line may convey the coolant away from the needle 202 after it has been used to cool the needle 202. The supply cable 206 may also include one or more wires that may be provide signals from one or more temperature sensors, thermocouples, flow sensors, or other sensors that may provide information regarding operating characteristics of the ablation probe 200. The needle 202 may include one or more temperature sensors, such as thermocouples, that may measure temperatures of the needle. The needle 202 may include, for example, a first thermocouple 208, a second thermocouple 210, and a third thermocouple 212 positioned along the needle 202 at different longitudinal positions. The first thermocouple 208, the second thermocouple 210, and the third thermocouple 212 may each cause a temperature signal to be generated and transferred to a printed circuit board assembly (PCBA) along a suitable wire in or on the needle 202. In other examples, the needle 202 may include more or less than three thermocouples or other temperature sensors.

The supply cable 206 may be an elongated tube or conduit and each of the power line, the coolant supply line, the coolant return line, and other wires and lines may extend within an outer shell of the supply cable 206. A length of the supply cable 206 may allow a user of the ablation probe 200 to position the needle 202 of the ablation probe 200 as desired and to allow the patient to be moved in and/or out of an imaging device during a treatment. For example, a location of the needle 202 may be determined using CT, MRI, Ultrasound, or other imaging devices before, during, or after treatment is performed. Thus, the supply cable 206 may be sufficiently long to allow these diagnostic procedures to be performed. In some examples, the supply cable 206 is at least 12 feet in length. In other examples, the supply cable 206 is at least 10 feet in length. In other examples, other lengths may be used.

The handle 204 may connect and/or operably couple the supply cable 206 to the needle 202. The power signal, the coolant, and/or sensor signals may be conveyed or delivered from the supply cable 206 to the needle 202 through the handle 204. The handle 204 may include a housing that may enclose various aspects such as fluid connections, electrical connections, electrical components, and the like. The handle 204 may be configured to orient the needle 202 in a desired alignment relative to the longitudinal axis of the supply cable 206. In the example shown, the handle 204 is a right-angle handle. The handle 204 orients the needle 202 at about 90 degrees (or substantially perpendicular) to the longitudinal axis of the supply cable 206. In other examples, the handle 204 may orient the needle 202 at a different angle relative to the longitudinal axis of the supply cable 206.

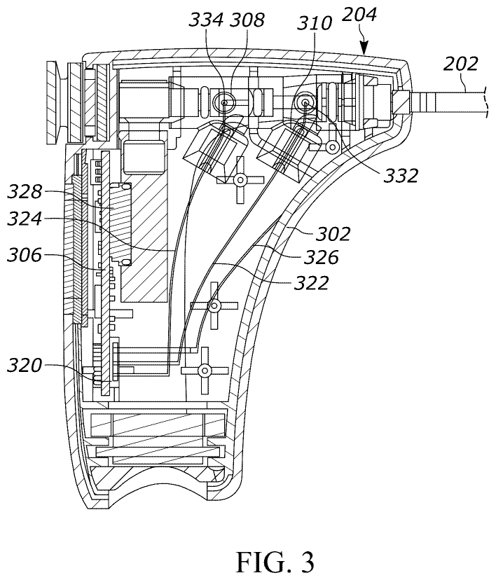

Turning now to FIG. 3, a view of the handle 204 is shown. In this example, the handle 204 is shown with a top portion of the housing removed so as to illustrate internal structure and aspects of the ablation probe 200. As shown, the handle 204 may include a housing 302. The housing 302 may be a shell or outer wall that may enclose various aspects of the ablation probe 200. The housing 302 may enclose a connection 304 of the power line in the supply cable 206 to the needle 202. The housing 302 may also enclose a printed circuit board assembly (PCBA) 306. The housing may also enclose a coolant supply connector 308 that may fluidly couple a coolant supply line from the supply cable 206 to a coolant flow path in the needle 202. The housing 302 may also enclose a coolant return connector 310 that may fluidly couple the coolant return line to the coolant flow path in the needle 202.

The PCBA 306 may include various electronic components to improve the functionality of the microwave ablation probe 200. The PCBA 306 may include an analog to digital converter 320. Sensor wires may be connected to the analog to digital converter 320. The handle 204 may include a coolant return wire 322 that electrically couples a coolant return thermocouple 332 to the analog to digital converter 320. The handle 204 may also include a coolant supply wire 324 that electrically couples a coolant supply thermocouple 334 to the analog to digital converter. The handle 204 may also include multiple needle thermocouple wires 336 that may electrically connect the thermocouples located on or in the needle 202 to the analog to digital converter.

The analog to digital converter may convert the analog signals from the various thermocouples to digital signals. In one example, a Max11410A analog to digital converter manufactured by Analog devices may be used. In another example, an analog to digital converter such as a high-speed, 16-bit, dual channel analog to digital converter manufactured by Texas Instruments may be used. In other examples, other analog to digital converters may be used. The analog to digital converter may take an analog voltage as an input and convert such input to an output that is a digital number corresponding to the voltage level. The digital signals may then be transferred from the handle 204 to the ablation console. The digital signals can be send with improved accuracy, reliability, and quality as compared to the analog signals. Existing ablation probes often transmit the analog temperature signals from the thermocouples all the way to an ablation console along the supply cable 206. Since such supply cables are often very long, such analog signals are susceptible to noise, distortion, interferences, or other signal loss. Thus, by converting the temperature signals to digital signals in the handle, the analog signals from the thermocouples are only transmitted a very short distance in the ablation probes of the present disclosure. In addition, the digital signals from multiple thermocouples may be transmitted to the ablation console along a single digital transmission wire in the supply cable 206. This also reduces a likelihood damage and may decrease the cost over existing ablation probes.

The PCBA 306 may also include an electrically erasable programmable read-only memory (EEPROM) device 328. The EEPROM device 328 may allow information to be stored and then accessed for later use. The EEPROM device 328 may be coupled to the ablation console 102 via the supply cable 206. The EEPROM device 328 may store various pieces of information. In one example, an EEPROM such as a 2-Kbit, 400 kHz EEPROM manufactured by ST Microelectronics may be used. In other examples, other EEPROM devices may be used. The EEPROM device 328 may store quality assurance information and/or testing information, for example. This information may include information regarding the manufacture and/or testing that was performed to verify the proper function of the device. Other information may also be stored such as probe characteristic information that may describe a model no., a needle size (diameter), an ablation zone size, an ablation zone shape, and/or recommended operating parameters. Still further, the EEPROM device 328 may store usage information that may indicate whether a probe has been used, a designated patient, or a designated treatment for the probe.

When the ablation probe 200 is coupled to the ablation console 102, the information stored on the EEPROM device 328 may be accessed or obtained by the console 102. The console 102 may compare such information to information such as a treatment plan for a particular treatment. The console 102 may also verify whether the probe has been previously used. The console 102 may alert the user if the anticipated information does not match with the information obtained from the EEPROM device 328. For example, the console 102 may alert the user that the probe 200 was previously used. In another examples, the console 102 may alert the user that the patient indicated in the treatment plan does not indicate the designated patient in the EEPROM. In still other examples, the console 102 may lock out or prevent use of the probe 200 until the usage information in the EEPROM device 328 matches the information in the treatment plan. In this manner, human error during ablation treatments can be reduced and/or eliminated.

The housing 302 of the ablation probe 200 may be made of suitable material such as a plastic, polymer, composite, or the like. The housing 302 may be formed to hold the various aspects (such as those previously described) in a desired position in the handle 204. Since the housing 302 may enclose one or more electronic components, such as the PCBA 306, it may be desirable to configure the housing to be waterproof. It may be desirable for the housing to resist and/or prevent the intrusion of external fluids. Therefore, the handle 204 may be sealed to resist the intrusion of external fluids. While not all aspects of the ablation probe 200 are listed here, other elements may also be enclosed in the housing 302.

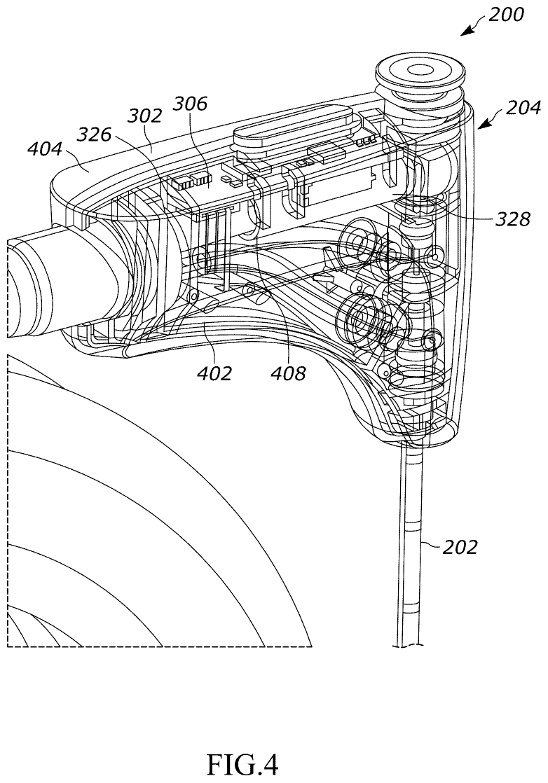

The handle 204 may be made of a top portion 402 and a bottom portion 404. An example bottom portion 404 of housing 302 is shown in FIG. 3. An example of the housing 302 in which the top portion 402 is connected to the bottom portion 404 of housing 302 is shown in FIG. 4. The bottom portion 404 of housing 302 may be connected to the top portion 402 to seal out the intrusion of fluids. In some examples, the top portion may be ultra-sonically welded to the bottom portion. In other examples, other suitable joining methods may be used such as bonding with adhesive, welding, fasteners, and the like.

As further shown, in FIG. 4, the PCBA may be positioned along an upper side of the housing 302. The PCBA may be positioned between the power cable 408 and the outer wall of the housing 302. In other examples, the PCBA may be positioned at other locations in the housing 302.

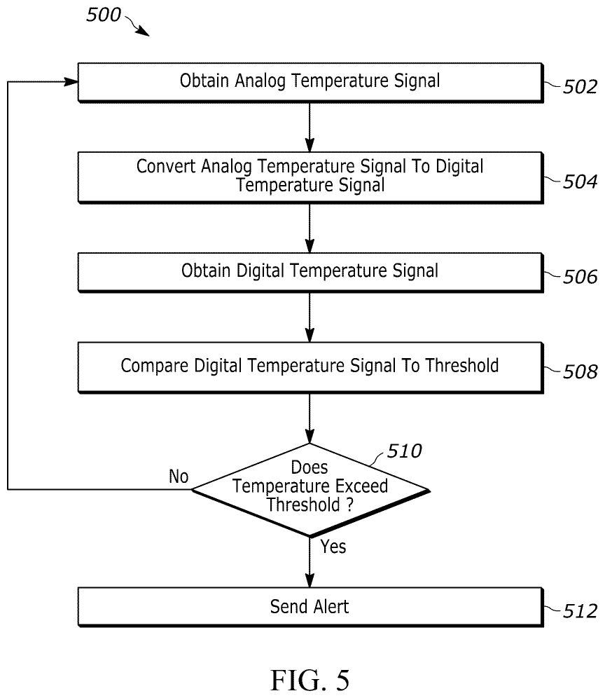

Referring now to FIG. 5, an example method 500 of performing an ablation treatment is shown. The method 500 may be performed using elements of the microwave ablation probe 200 and/or the microwave ablation apparatus 100. The method 500 is described below with reference to the microwave ablation apparatus 100 and the microwave ablation probe 200. It should be appreciated that other apparatuses or variations of the microwave ablation probe 200 and/or the microwave ablation apparatus 100 may also be used.

The method 500 may begin at step 502. At step 502, the PCBA in the handle 204 of the ablation probe 200 may obtain an analog temperature signal. The analog temperature signal may be obtained from one of the thermocouples described above. The thermocouples may be located in the handle 204 or at the needle 202. The thermocouples may provide information regarding a temperature of the coolant or the needle as previously described.

The analog temperature signal may be converted to a digital temperature signal at step 504. The conversion may be performed using the analog to digital converter 320, for example. This conversion is performed in the handle 204 so that the analog temperature signal needs to be transmitted less than 24 inches. This improves the reliability, accuracy, and quality of the temperature measurements due to the inherent risk of noise, interference, and signal loss of analog signals that may occur particularly, if the analog signals are transmitted over a long distance.

The ablation console 102 may obtain the digital temperature signal at step 506. The ablation console 102 may obtain the digital temperature signal from the handle 204 of the probe 200 via the supply cable 206. The distance that the digital temperature signal is transmitted may be more than 10 feet in length. Since this is a digital signal that is transmitted rather than analog, the reliability, quality, and accuracy of the signal is much improved over existing probe designs and methods.

At step 508, the ablation console 102 (that includes at least one computing device) may compare the temperature indicated by the digital temperature signal to a temperature threshold. The temperature threshold may correspond to various temperature thresholds depending on the temperature in question. In some examples, the temperature may indicate a predetermined temperature threshold of return coolant. In other examples, the temperature threshold may indicate a predetermined temperature threshold of supply coolant. In still other examples, the temperature threshold may correspond to a temperature threshold of the needle. The temperature thresholds may be input by a user into the ablation console 102. Alternatively, the temperature thresholds may be obtained by the ablation console 102 from a medical or clinical database or from a treatment plan.

At step 510, the ablation console 102 may determine whether the temperature exceeds the temperature threshold. If the temperature does not exceed the temperature threshold, the method may return to step 502 whereby the steps 502 through 510 may be re-performed continuously or periodically to monitor the various temperatures of the ablation probe 200. If the temperature exceeds the temperature threshold, the method moves to step 512.

At step 512, the ablation console 102 may send an alert to the user. The ablation console 102 may send an alert via a user interface and/or may communicate the alert using visual or audible indicators. The alert may indicate to the user that the coolant is running too hot and may need to be replaced. The alert may indicate to the user that the needle is running too hot and the power level to the microwave antenna may need to be adjusted. Still further, the temperature may indicate that the probe 200 is not performing as anticipated and the treatment should be adjusted and/or delayed. In other examples, the ablation console 102 may provide other alerts.

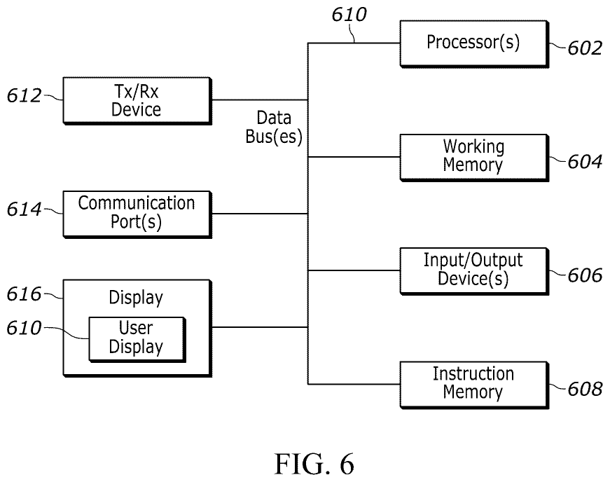

Referring now to FIG. 6, an example computing device 600 is shown. The microwave ablation apparatus 100 may include one or more computing devices 600. For example, the microwave ablation console 102 may have the elements shown in FIG. 6. The methods of the present disclosure, such as method 500, may be performed, or steps of such methods may be performed, by a computing device 600.

As shown, the computing device 600 may include one or more processors 602, working memory 604, one or more input/output devices 606, instruction memory 608, a transceiver 612, one or more communication ports 614, and a display 616, all operatively coupled to one or more data buses 610. Data buses 610 allow for communication among the various devices. Data buses 610 can include wired, or wireless, communication channels.

Processors 602 can include one or more distinct processors, each having one or more cores. Each of the distinct processors can have the same or different structure. Processors 602 can include one or more central processing units (CPUs), one or more graphics processing units (GPUs), application specific integrated circuits (ASICs), digital signal processors (DSPs), and the like.

Processors 602 can be configured to perform a certain function or operation by executing code, stored on instruction memory 608, embodying the function or operation. For example, processors 602 can be configured to perform one or more of any function, step, method, or operation disclosed herein.

Instruction memory 608 can store instructions that can be accessed (e.g., read) and executed by processors 602. For example, instruction memory 608 can be a non-transitory, computer-readable storage medium such as a read-only memory (ROM), an electrically erasable programmable read-only memory (EEPROM), flash memory, a removable disk, CD-ROM, any non-volatile memory, or any other suitable memory.

Processors 602 can store data to, and read data from, working memory 604. For example, processors 602 can store a working set of instructions to working memory 604, such as instructions loaded from instruction memory 608. Processors 602 can also use working memory 604 to store dynamic data created during the operation of microwave ablation console 102. Working memory 604 can be a random access memory (RAM) such as a static random access memory (SRAM) or dynamic random access memory (DRAM), or any other suitable memory.

Input-output devices 606 can include any suitable device that allows for data input or output. For example, input-output devices 606 can include one or more of a keyboard, a touchpad, a mouse, a stylus, a touchscreen, a physical button, a speaker, a microphone, or any other suitable input or output device.

Communication port(s) 614 can include, for example, a serial port such as a universal asynchronous receiver/transmitter (UART) connection, a Universal Serial Bus (USB) connection, or any other suitable communication port or connection. In some examples, communication port(s) 614 allows for the programming of executable instructions in instruction memory 608. In some examples, communication port(s) 614 allow for the transfer (e.g., uploading or downloading) of data, such as temperature information, usage information and the like.

Display 616 can display a user interface 618. User interfaces 618 can enable user interaction with the microwave ablation console 102. For example, user interface 618 can be a user interface that allows an operator to interact, communicate, control and/or modify different messages, settings, or features that may be presented or otherwise displayed to a user. For example, alerts may be communicated using the user interface 618. The user interface 618 can include a slider bar, dialogue box, or other input field that allows the user to control, communicate or modify a setting, limitation or input that is used in an ablation treatment. In addition, the user interface 618 can include one or more input fields or controls that allow a user to modify or control optional features or customizable aspects of the microwave ablation console 102 and/or the operating parameters of the microwave ablation apparatus 100. In some examples, a user can interact with user interface 618 by engaging input-output devices 606. In some examples, display 616 can be a touchscreen, where user interface 618 is displayed on the touchscreen. In other examples, display 616 can be a computer display that can be interacted with using a mouse or keyboard.

Transceiver 612 allows for communication with a network. In some examples, transceiver 612 is selected based on the type of communication network ablation console 102 will be operating in. Processor(s) 602 is operable to receive data from, or send data to, a network, such as wired or wireless network that couples the elements of the microwave ablation apparatus 100.

The following is a list of non-limiting illustrative embodiments disclosed herein:

Illustrative embodiment 1: A microwave ablation probe comprising: a handle configured to operably couple a supply cable to a needle; and a printed circuit board assembly (PCBA) positioned in the handle, the PCBA comprising an analog to digital converter and an electrically erasable programmable read-only memory (EEPROM) device.

Illustrative embodiment 2: The microwave ablation probe of illustrative embodiment 1, wherein the PCBA is operatively coupled to a plurality of thermocouples positioned at a plurality of locations in the microwave ablation probe.

Illustrative embodiment 3: The microwave ablation probe of illustrative embodiment 2, wherein the plurality of thermocouples comprises a first thermocouple located in the handle and configured to determine a temperature of a supply coolant, and a second thermocouple located in the handle and configured to determine a temperature of a return coolant.

Illustrative embodiment 4: The microwave ablation probe of illustrative embodiment 3, wherein the plurality of thermocouples comprises a third thermocouple located in or on the needle and configured to determine a temperature of the needle.

Illustrative embodiment 5: The microwave ablation probe of any of illustrative embodiments 2 to 4, wherein the analogue to digital converted is configured to receive a plurality of analog temperature signals from the plurality of thermocouples and send a digital temperature signal based on the plurality of analog temperature signals.

Illustrative embodiment 6: The microwave ablation probe of any of illustrative embodiments 2 to 5, further comprising: a plurality of thermocouple wires each operably connecting a respective thermocouple of the plurality of thermocouples to the PCBA; and a digital signal wire operably connecting the PCBA to an ablation console.

Illustrative embodiment 7: The microwave ablation probe of any of illustrative embodiments 1 to 6, wherein the EEPROM device is configured to store probe information characterizing a treatment performed using the microwave ablation probe.

Illustrative embodiment 8: The microwave ablation probe of any of illustrative embodiments 1 to 7, wherein the EEPROM device is configured to store calibration information characterizing a calibration of or testing performed for the microwave ablation probe.

Illustrative embodiment 9: A microwave ablation apparatus comprising: the microwave ablation probe of any of illustrative embodiments 1 to 8; an ablation console comprising at least one computing device; and the supply cable operably connecting the microwave ablation probe to the ablation console.

Illustrative embodiment 10: The microwave ablation apparatus of illustrative embodiment 9, wherein the at least one computing device is configured to: obtain a coolant temperature signal from the PCBA characterizing a temperature of a coolant in the handle; and send a coolant temperature alert when the temperature exceeds a predetermined coolant temperature threshold.

Illustrative embodiment 11: The microwave ablation apparatus of illustrative embodiment 10, wherein the coolant temperature signal is a digital signal obtained from the analog to digital converter.

Illustrative embodiment 12: The microwave ablation apparatus of any of illustrative embodiments 9 to 11, wherein the at least one computing device is configured to: obtain a needle temperature signal from the PCBA characterizing a temperature of the needle; and send a needle temperature alert when the temperature exceeds a predetermined needle temperature threshold.

Illustrative embodiment 13: The microwave ablation apparatus of illustrative embodiment 12, wherein the needle temperature signal is a digital signal obtained from the analog to digital converter.

Illustrative embodiment 14: The microwave ablation apparatus of any of illustrative embodiments 9 to 13, wherein the at least one computing device is configured to: obtain usage information from the EEPROM device; and compare the usage information to treatment information; and send a usage alert when the usage information does not correspond to the treatment information.

Illustrative embodiment 15: A microwave ablation apparatus comprising: an ablation console comprising at least one computing device; and a microwave ablation probe coupled to the ablation console, the microwave ablation probe comprising: a handle configured to operably couple a supply cable to a needle; and a printed circuit board assembly (PCBA) positioned in the handle, the PCBA comprising an analog to digital converter and an electrically erasable programmable read-only memory (EEPROM) device.

Illustrative embodiment 16: A method comprising: obtaining an analog temperature signal from a microwave ablation probe; converting the analog temperature signal to a digital temperature signal in the handle of the microwave ablation probe; and sending the digital temperature signal to a microwave ablation console.

Illustrative embodiment 17: The method of illustrative embodiment 16, wherein the analog temperature signal characterizes a temperature of coolant in the handle of the microwave ablation probe.

Illustrative embodiment 18: The method of any of illustrative embodiments 16 or 17, wherein the analog temperature signal characterizes a temperature of a needle of the microwave ablation probe.

Illustrative embodiment 19: The method of any of illustrative embodiments 16 to 18, further comprising sending usage information from an electrically erasable programmable read-only memory (EEPROM) device located in the handle of the microwave ablation probe.

Illustrative embodiment 20: The method of any of illustrative embodiments 16 to 19, further comprising sending a temperature alert when the temperature exceeds a predetermined temperature threshold.

The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A microwave ablation probe comprising:
a handle configured to operably couple a supply cable to a needle; and
a printed circuit board assembly (PCBA) positioned in the handle, the PCBA comprising an analog to digital converter and an electrically erasable programmable read-only memory (EEPROM) device.

2. The microwave ablation probe of claim 1, wherein the PCBA is operatively coupled to a plurality of thermocouples positioned at a plurality of locations in the microwave ablation probe.

3. The microwave ablation probe of claim 2, wherein the plurality of thermocouples comprises a first thermocouple located in the handle and configured to determine a temperature of a supply coolant, and a second thermocouple located in the handle and configured to determine a temperature of a return coolant.

4. The microwave ablation probe of claim 3, wherein the plurality of thermocouples comprises a third thermocouple located in or on the needle and configured to determine a temperature of the needle.

5. The microwave ablation probe of claim 2, wherein the analogue to digital converter is configured to receive a plurality of analog temperature signals from the plurality of thermocouples and send a digital temperature signal based on the plurality of analog temperature signals.

6. The microwave ablation probe of claim 2, further comprising:
a plurality of thermocouple wires each operably connecting a respective thermocouple of the plurality of thermocouples to the PCBA; and
a digital signal wire operably connecting the PCBA to an ablation console.

7. The microwave ablation probe of claim 1, wherein the EEPROM device is configured to store probe information characterizing a treatment performed using the microwave ablation probe.

8. The microwave ablation probe of claim 1, wherein the EEPROM device is configured to store calibration information characterizing a calibration of or testing performed for the microwave ablation probe.

9. A microwave ablation apparatus comprising:
the microwave ablation probe of claim 1;
an ablation console comprising at least one computing device; and
the supply cable operably connecting the microwave ablation probe to the ablation console.

10. The microwave ablation apparatus of claim 9, wherein the at least one computing device is configured to:
obtain a coolant temperature signal from the PCBA characterizing a temperature of a coolant in the handle; and
send a coolant temperature alert when the temperature exceeds a predetermined coolant temperature threshold.

11. The microwave ablation apparatus of claim 10, wherein the coolant temperature signal is a digital signal obtained from the analog to digital converter.

12. The microwave ablation apparatus of claim 9, wherein the at least one computing device is configured to:
obtain a needle temperature signal from the PCBA characterizing a temperature of the needle; and
send a needle temperature alert when the temperature exceeds a predetermined needle temperature threshold.

13. The microwave ablation apparatus of claim 12, wherein the needle temperature signal is a digital signal obtained from the analog to digital converter.

14. The microwave ablation apparatus of claim 9, wherein the at least one computing device is configured to:
obtain usage information from the EEPROM device;
compare the usage information to treatment information; and
send a usage alert when the usage information does not correspond to the treatment information.

15. A microwave ablation apparatus comprising:
an ablation console comprising at least one computing device; and
a microwave ablation probe coupled to the ablation console, the microwave ablation probe comprising:
a handle configured to operably couple a supply cable to a needle; and
a printed circuit board assembly (PCBA) positioned in the handle, the PCBA comprising an analog to digital converter and an electrically erasable programmable read-only memory (EEPROM) device.

16. A method comprising:
obtaining an analog temperature signal from a microwave ablation probe;
converting the analog temperature signal to a digital temperature signal in the handle of the microwave ablation probe; and
sending the digital temperature signal to a microwave ablation console.

17. The method of claim 16, wherein the analog temperature signal characterizes a temperature of coolant in the handle of the microwave ablation probe.

18. The method of claim 16, wherein the analog temperature signal characterizes a temperature of a needle of the microwave ablation probe.

19. The method of claim 16, further comprising sending usage information from an electrically erasable programmable read-only memory (EEPROM) device located in the handle of the microwave ablation probe.

20. The method of claim 16, further comprising sending a temperature alert when the temperature exceeds a predetermined temperature threshold.
